(19) United States
(12) Patent Application Publication
(10) Pub. No.: US 2019/0076482 A1
(43) Pub. Date: Mar. 14, 2019

(54) ENHANCED ORGANOID FORMATION AND INTESTINAL STEM CELL RENEWAL

(71) Applicant: THE JOHNS HOPKINS UNIVERSITY, Baltimore, MD (US)

(72) Inventors: Linda Smith-Resar, Stevenson, MD (US); David Huso, Parkton, MD (US); Lingling Xian, Timonium, MD (US)

(21) Appl. No.: 16/084,641

(22) PCT Filed: Mar. 13, 2017
(86) PCT No.: PCT/US2017/022002

§ 371 Date: Sep. 13, 2018

Related U.S. Application Data
(60) Provisional application No. 62/308,288, filed on Mar. 15, 2016.

(51) Int. Cl. A61K 35/38; A61K 38/17; C12N 5/071; C12N 15/85

(52) U.S. Cl. CPC A61K 35/38; A61K 38/1709; C12N 5/0679; C12N 2513/00; A61K 38/177; C12N 2501/60; C12N 2510/00; C12N 15/8509

(57) ABSTRACT

High mobility group A1 (Hmga1) chromatin remodeling proteins are enriched in intestinal stem cells (ISCs), although their function in this setting was unknown. The present invention describes a method of producing a genetically modified organoid comprising the following steps: isolating a crypt of the small intestine; culturing a portion of the crypt in a culture medium; forming an organoid; and transducing the organoid with a vector comprising Hmga1. Methods using genetically modified organoids to treat or prevent injuries including cancer are described.

### REFERENCE TO RELATED APPLICATIONS

This application claims the benefit of U.S. Provisional Patent Application No. 62/308,288, filed on Mar. 15, 2016, both of which are hereby incorporated by reference for all purposes as if fully set forth herein.

### STATEMENT OF GOVERNMENTAL INTEREST

This invention was made with government support under grant nos. R21CA149550 and R03CA182679 from the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Mar. 2, 2017, is named P14031-02_SL.txt and is 11,963 bytes in size.

### BACKGROUND OF THE INVENTION

The high-mobility group (HMG) proteins are low-molecular weight nuclear proteins and among the most abundant nonhistone chromatin binding factors found in the nucleus of cancer cells. The A subfamily of HMG proteins (i.e. HMGA1a, HMGa1b, HMGA1c, and HMGA2) interacts with the minor groove of many AT-rich promoters and enhancers and plays key roles in chromatin architecture and gene transcription control. HMGA proteins do not appear to alter gene transcriptional activity alone; rather, they alter chromatin structure and recruit additional transcription factors to DNA. They are therefore considered “architectural transcription factors”. By a complex network of protein-DNA and protein-protein interactions, they organize chromatin into a structure required to execute gene transcription. In physiological conditions, HMGA proteins are expressed at high level during embryogenesis while their expression becomes low to undetectable in adult differentiated tissues. High HMGA expression post-natally is associated with poorly differentiated/refractory, “stem-like” cancers such as human carcinomas of thyroid, colon, prostate, pancreas, cervix, ovary, breast, and blood. HMGA1 is also highly expressed in embryonic stem cells, cancer stem cells and adult stem cells, such as hematopoietic stem cells and intestinal cells.

Intestinal stem cells (ISCs) provide a paradigm for studying adult stem cell function due to their exceptional self-renewal potential and repetitive structural organization. Indeed, the intestinal lining is among the most highly regenerative tissues, renewing itself every 3-5 days to protect the gut from pathogens and maintain nutrient intake essential for life. Over the past decade, a population of self-renewing, columnar epithelial cells located at the base of the intestinal crypts has been identified and characterized as ISCs. They are marked by the serpentine receptor, leucine-rich repeat containing G-protein-coupled receptor 5 (Lgr5), which mediates Wnt signaling cues from the niche. Lineage tracing experiments demonstrate that these ISCs are responsible for the exuberant regeneration and tissue homeostasis in intestinal epithelium. Despite extensive study, the molecular mechanisms that govern their behavior are only beginning to be elucidated. Previous work also demonstrates that aberrant expression or mutation of key regulators of ISCs leads to neoplastic growth and intestinal carcinogenesis.

Emerging evidence highlights the central role for chromatin structure and chromatin binding proteins in maintaining stem cell properties. In fact, recent work found that the High Mobility Group A1 chromatin remodeling proteins (HMGA1, formerly HMG-I/Y) regulate stem cell properties in cancer, although their role in normal development has remained elusive. The HMGA1 gene encodes the HMGA1 a and HMGA1b isoforms, which function as architectural transcription factors that bend DNA and recruit other transcriptional complexes to regulatory regions throughout the genome. HMGA1 is highly expressed during embryogenesis, with high levels in normal embryonic stem cells. Postnatally, HMGA1 is expressed in adult stem cells, such as hematopoietic and intestinal stem cells, but absent or barely detectable in mature, differentiated tissues. In cancer, HMGA1 becomes aberrantly expressed through oncogenic transcription factors and epigenetic alterations, or in rare cases, chromosomal translocation events. Moreover, HMGA1 is overexpressed in all high-grade or poorly-differentiated cancers studied to date, and high levels portend a poor prognosis in diverse tumors. In murine tumor xenografts, HMGA1 drives tumor progression and cancer stem cell properties, at least in part, by inducing stem cell transcriptional networks. In human embryonic stem cells, HMGA1 maintains a de-differentiated state by up-regulating genes involved in sternness and pluripotency. Moreover, HMGA1 is required for reprogramming somatic cells to induced pluripotent stem cells by the Yamanaka factors; disrupting HMGA1 expression or function prevents the derivation of fully reprogrammed cells. Given its dual role in normal development and cancer, further studies to dissect its function in each setting are needed to determine the therapeutic potential of targeting HMGA 1 in cancer or harnessing HMGA1 function for tissue regeneration. Tissue regeneration has many applications in medicine including enhanced healing of wounds and treatment of complicated injuries that require tissue growth. A commercial need exists for new methods of creating tissue in-vitro that may be placed back into a subject for purposes of wound treatment or replacement of tissues damaged by disease or other forms of injury.

### SUMMARY OF THE INVENTION

One embodiment of the present invention is a method of producing a genetically modified organoid comprising the following steps: isolating a crypt of the intestine; culturing at least a portion of the crypt in a culture medium; forming an organoid; and transducing the organoid with a vector expressing a Hmga1 protein or functional part thereof. Examples of suitable vectors includes retroviruses, lentiviruses, adenoviruses, and/or adeno-associated viruses, as examples. One effective vector is the FUGW-Hmga1 lentiviral vector, for example. The proximal small intestine is a region that has yield crypt cells and/or intestinal cells with efficient transduction that are suitable for the present invention. Other cells suitable for the present invention including cells of the large intestine, for example.

Another embodiment of the present invention is a genetically modified organoid comprising: a cell of the intestine comprising a vector expressing an Hmga1 protein, or functional part thereof. The vector may be integrated into the genome of the cells. Any suitable vector maybe used in the invention including a FUGW-Hmga1 lentiviral vector. Such a genetically modified organoid of the present invention has enhanced Wnt signaling when compared to a reference organoid substantially free of a vector expressing a Hmga 1 protein or functional part thereof. The enhanced Wnt signaling results in enhanced expression of gene downstream of WntTcf4/β-catenin. The genetically modified organoids of the present invention have increased numbers of Paneth when compared to a reference organoid substantially free of a vector expressing a Hmga1 protein or functional part thereof. Suitable organoids used in the present invention comprise Sox 9 and the Hmga 1 proteins expressed from the vector up-regulates Sox 9 expression when compared to a reference organoid.

Another embodiment of the present invention is a method of treating or preventing an injury in a subject comprising the following steps: providing a subject with an intestinal injury; and placing an organoid comprising a vector expressing a Hmga1 protein, or functional part thereof, adjacent to the site of the intestinal injury. Suitable organoids used in the present invention comprise an intestine cell comprising a vector such as lentiviral vector, for example. The vector may be integrated in a genome of the intestine cell. The organoids of the present invention have increased number of Paneth when compared to a reference organoid. Placing the organoids of the present invention adjacent to the site of an intestinal injury will increase the number of Paneth adjacent to the site of intestinal injury. In addition a subject comprising an organoid of the present invention within their intestine has enhanced intestinal stem cell maintenance compared to a reference subject that has not undergone a method of the present invention. A subject of the present invention may have been administered chemotherapy or radiation as examples of treatments that may result in intestinal injury.

Another embodiment of the present invention is a method of screening a pharmaceutical agent comprising: providing a first organoid comprising a vector expressing a Hmga1 protein or functional part thereof applying an agent to the first organoid; and identifying those agents that inhibit the expression or activity of the Hmga1 protein, or functional part thereof, in the first organoid. Suitable agents include chemicals, proteins, peptides, nucleic acids sequences, or combination thereof. Methods of the present invention may further provide a reference organoid that is substantially free of a vector expressing a Hmga 1 protein, or functional part thereof, and the agent is applied to the reference organoid.

Another embodiment of the present invention is a method of treating or preventing intestinal cancer in a subject comprising administering to the subject an effective amount of a Hmga1 inhibitor or a pharmaceutically acceptable salt, solvate, or stereoisomer thereof. Suitable Hmga1 inhibitor include chemicals, peptides, antibodies, shRNAs, nucleic acids, or combination thereof. An example of an intestinal cancer is colorectal neoplasia.

### BRIEF DESCRIPTION OF THE DRAWINGS

FIG. 1A to 1G illustrates the Hmga1 protein localization.

FIG. 2A to 2E illustrates organoid characteristics of genetically modified organoids formed from isolated crypts of the proximal small intestine.

FIG. 3A to 3B illustrates organoid characterization.

FIG. 4A to 4F illustrates Hmga1 induces ISC self-renewal through Wnt/β-catenin signaling.

FIG. 5A-5I illustrates Hmga1 organoids cultured in the absence of the Wnt receptor agonist, R-spondin.

FIGS. 6 and 7 illustrate a model of Hmga 1 in normal homeostasis and reprogramming to colorectal neoplasia.

FIG. 8A to 8C illustrates Hmga1 transgenic mouse organoids exhibit enhanced sensitivity to Wnt and amplify genes encoding Wnt agonist receptors and downstream effectors. (a) Increased number of cyst-like structures from Hmga1 transgenic mouse organoids after exposure to Wnt3a (10 ug/ml). *p,0.05 (b) Greater diameter of cyst-like structures from Hmga1 transgenic mouse organoids after exposure to Wnt3a (10 ug/ml). *P,0.05 (c) Enhanced expression of Wnt/Tcf4/β-catenin genes in organoids derived from Hmga1 trangenic mice as compared to WT mice. *P<0.05.

FIG. 9 illustrates organoids from WT or Hmga1 transgenic mice in the presence or absence of R-Spondin 1 (R-Spo1). Representing images of WT and Hmga1 organoids cultured with or without R-Spo 1 (1 ug/ml) at 6 and 12 days.

FIG. 10A to 10B illustrates Hmga1 directly induces SOX9 mRNA. (a) Sox9 expression in control organoids (FUGW) or WT organoids engineered to overexpress Hmga1 (FUGW-Hmga1). (b) Diagram of Hmga1 consensus DNA binding sites on the Sox9 promoter (MatInspector). The arrow denotes the transcription start site.

FIG. 11A to 11C illustrates Hmga1 transgenic crypt cells generate larger 3D organoids with enhanced bud formation. (a) Hmga1 expression was assessed 21 days after lentiviral transduction by qRT-PCR in the organoids derived from WT or Hmga1 transgenic mice at 4 months. Gapdh expression was used to control for loading. *P<0.05. (b) Hmga1 organoids were larger and generated more buds, as shown here by hematoxylin & eosin (H & E). Scale bar: 50 um. (c) Hmga1 expression in the WT organoids transduced with control lentivirus (FUGW-GFP, labeled FUGW) or lentivirus overexpression Hmga1 (FUGW-Hmga1-GFP, labeled FUGW-Hmga1) *P<0.05.

FIG. 12 illustrates Hmga1 immunohistochemical staining in organoids from WT or transgenic mice. Immunohistochemical (IHC) staining for Hmga1 (brown) in organoid sections derived from 4 month-old WT or Hmga1 transgenic mice. Black arrows indicate intense staining at the bud tips; the blue arrows indicate decreased or absent staining in more differentiated cells. Scale bar: 50 um.

FIG. 13A-13C illustrates silencing Hmga1 mRNA in organoids using 2 different lentiviral vectors targeting Hmga1 disrupts 3D organoid formation. (a) Hmga1 expression in WT organoids transduced by control lentivirus (TRC) or lentivirus encoding shRNA targeting Hmga1 (TRC-shHmga1) at day 21. *P<0.05. (b) Hmga1 expression in WT organoids transduced by a lentivirus encoding inducible shRNA targeting Hmga1 (pTRIPz-Hmga-shRNA) with or without doxycycline at day 5. (c) Hmga1 organoids transduced with lentivirus encoding shRNA targeting Hmga1 (4× magnification).

FIG. 14 illustrates Table 1 Wnt receptor agonist and Wnt/Tcf4/β-catenin genes. Figure discloses SEQ ID NOS 7-36, respectively, in order of appearance.

FIG. 15 illustrates Table 2 ChIP primers. Figure discloses SEQ ID NOS 37-42, respectively, in order of appearance.

FIG. 16 illustrates Table 3 Antibody list.

### DETAILED DESCRIPTION OF THE INVENTION

The inventors have demonstrated that transgenic mice overexpressing murine Hmga1 from the H-2Kb promoter and immunoglobulin u enhancer all succumb to lymphoid tumors; females also develop uterine sarcomas. In this model, the transgene was expressed in the intestines in addition to lymphoid cells and uterine tissue. The Hmga1 transgenics developed marked proliferative changes in the epithelium of the small and large intestine, with aberrant crypt formation and polyposis. In order to determine how Hmga1 disrupts tissue homeostasis in the intestines of transgenic mice and intestinal cancers overexpressing HMGA1, its expression and function was examined in another transgenic model and in intestinal organoids. It was discovered by the inventors that Hmga1 expands the ISC pool and Paneth cell niche in vivo. Hmga1 is essential for organization of ISCs into three-dimensional (3D) organoids that form crypt-like buds in vitro. It was also determined in the present invention that Hmga1 enhances ISC self-renewal and proliferation by amplifying Wnt/β-catenin signaling. Surprisingly, Hmga1 was determined to induce terminal differentiation of ISCs to Paneth cells by directly up-regulating Sox9. The present invention provided the first example of Hmga1 fostering terminal differentiation to establish a stem cell niche. Moreover, the present invention establishes that both TIMGA1 and SOX9 were positively correlated in human intestinal epithelium, and both become markedly up-regulated in colorectal cancer. These results of the present invention uncovered a novel role for Hmga1 in maintaining both the ISC pool and niche cells within intestinal crypts and suggest that this equilibrium is perturbed when Hmga1 becomes deregulated during carcinogenesis.

### Hmga1 is Expressed in ISCs and its Overexpression Drives Expansion of the ISC Compartment.

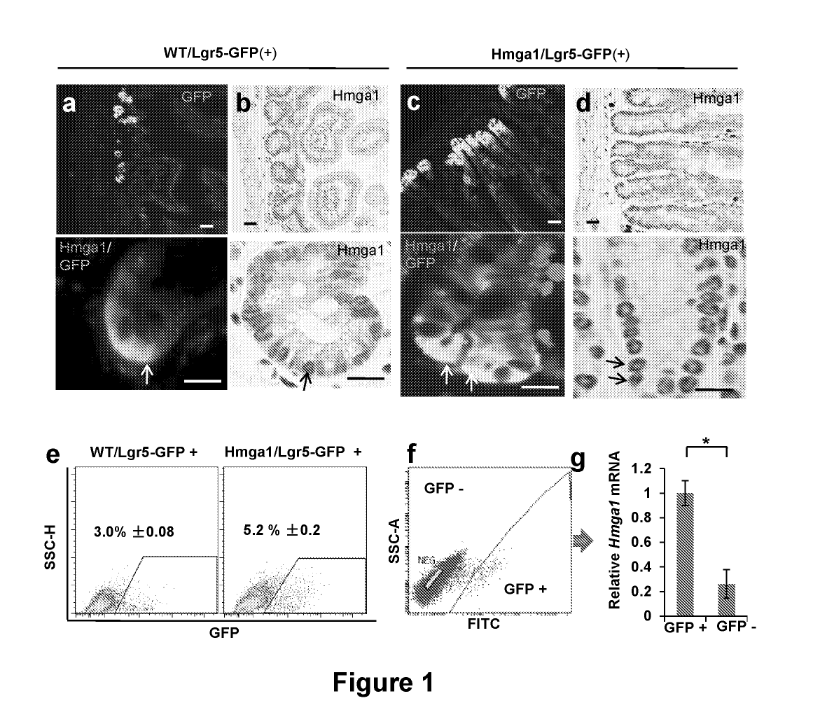

A prior gene expression profile study showed that Hmga1 is among the genes enriched in Lgr5+ ISCs. HMGA1 is also among the genes most highly expressed in diverse epithelial human cancers as compared to normal epithelium, including intestinal malignancies. The present invention has elucidated the functional role of Hmga1 in ISCs, both in normal intestinal epithelial homeostasis and in intestinal neoplasia. To this end, Hmga1 transgenic mice were crossed onto Lgr5-EGFP mice, which mark Lgr5+ ISCs with enhanced green fluorescent protein (EGFP). The Hmga1 transgene was driven by the H-2Kb promoter and μ enhancer, which confer transgene expression in intestinal crypt basilar cells, lymphoid cells and uterine tissue. In both Hmga1 transgenic and wildtype (WT) mice, Hmga1 protein localized to the nuclei of Lgr5+ ISCs (FIG. 1a-g). Interestingly, Lgr5+ cells extend further up the crypts in the Hmga1 transgenic mice as compared to WT mice (FIG. 1a, c), consistent with expansion in the ISC pool and enhanced self-renewal. The number of Lgr5+ cells/individual crypt by fluorescent stain (FIG. 1c) and the relative frequency of Lgr5+ISCs/crypt isolated by fluorescence activated cell sorting (FACS) were increased in the Hmga1 transgenic small intestine (FIG. 1e), further validating the expansion in ISCs. To determine whether Hmga1 gene expression accounts for the elevated protein levels in the Lgr5+ ISCs, the inventors assessed Hmga1 mRNA and found that it is increased by ˜4-fold in Lgr5+ ISCs isolated by fluorescence activated cell sorting (FACS) as compared to Lgr5− cells in WT mice (FIG. 1g).

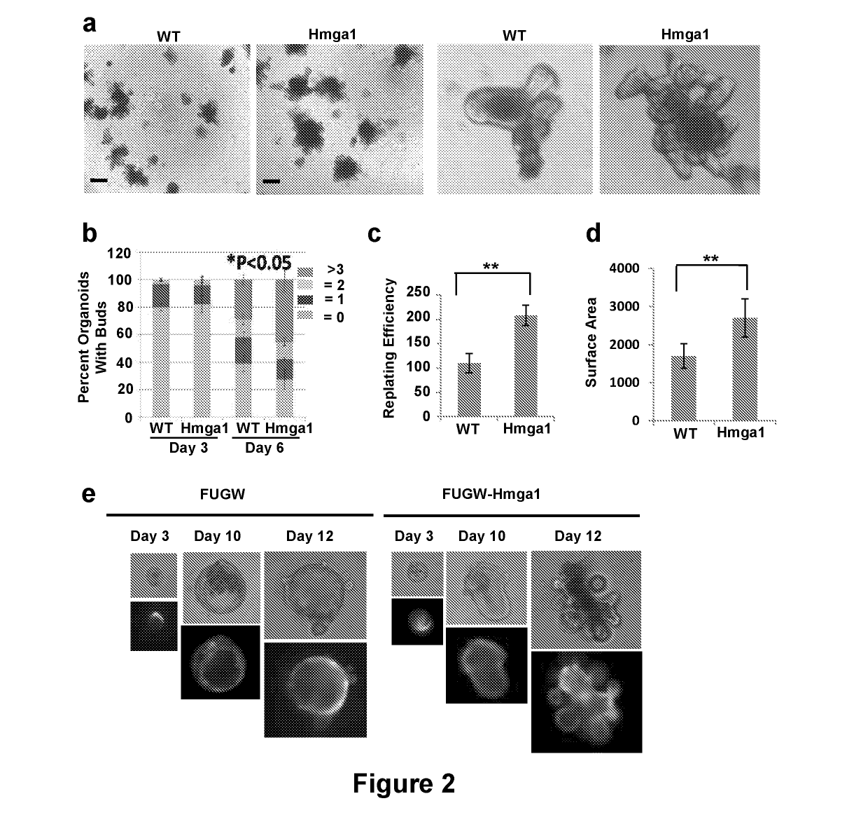

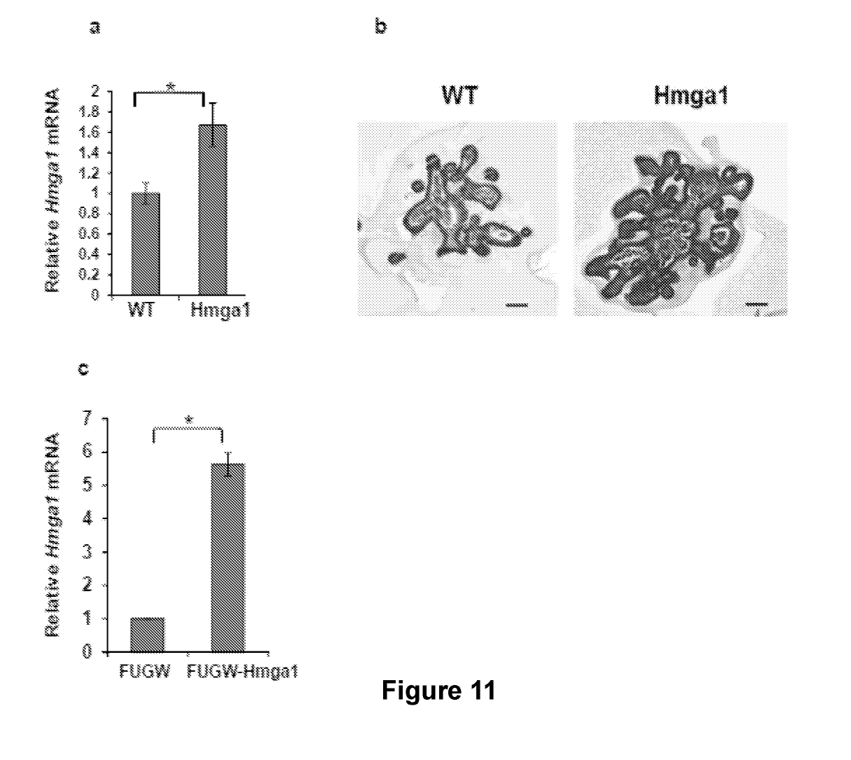

ISCs are regulated by factors from the stromal compartment in addition to intestinal epithelial cells. To define the role of Hmga1 in ISCs within the epithelial compartment, the inventors used organoids, an in vitro intestinal crypt culture model. Organoid buds are a surrogate for ISC function because they are comprised of crypt-like structures with ISCs on the tips; differentiated epithelial cells extend towards the luminal centers of the organoids. The inventors derived organoids from small intestinal epithelial crypt cells isolated from transgenic or WT mice and compared bud formation, surface area, and replating efficiency. Similar to the transgenic mouse intestine, the inventors found a dramatic increase in bud number per organoid and total organoid size in those derived from the Hmga1 transgenic mice as compared to WT controls after only 5 days in culture (FIG. 2a-b; FIG. 11a-b). There were significantly more Hmga1 organoids with >3 buds as compared to WT control organoids (FIG. 2b). The inventors also assessed replating efficiency by mechanically dispersing an equal number of organoids after 3-5 days in culture. There was a striking increase in both replating efficiency (>2-fold) and surface area/organoid in the Hmga1 cultures as compared to WT controls (FIG. 2c-d).

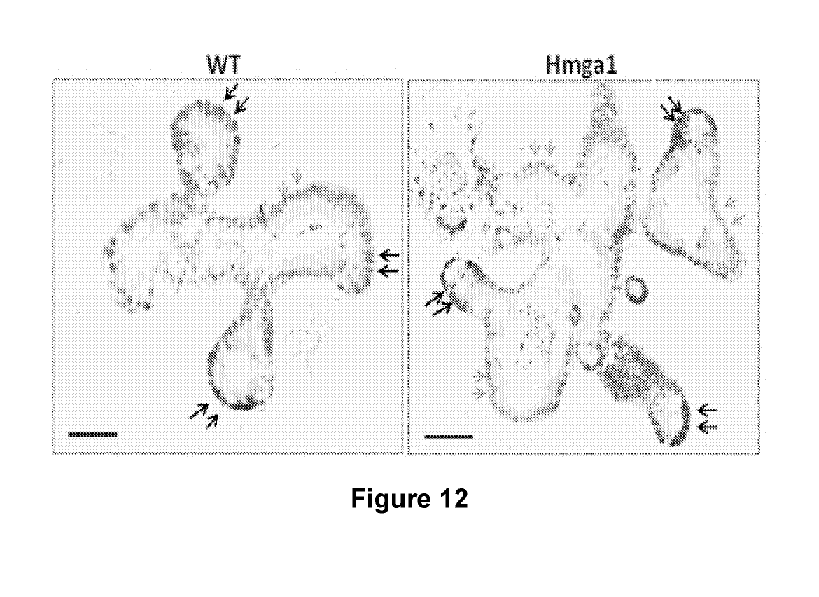

Because the inventors could not exclude the possibility that the enhanced ISC function in the Hmga1 organoids resulted from in vivo exposure to lymphoid or other cells with transgenic Hmga1 expression and downstream factors, the inventors also transduced WT organoids to overexpress Hmga1. The WT organoids engineered to overexpress Hmga1 exhibited a similar phenotype, with increased bud formation (FIG. 2e; FIG. 11c). Previous work demonstrated that forced expression of Hmga1 prevents differentiation in human embryonic stem cells. We therefore reasoned that Hmga1 expression would predominate at the crypt-like regions enriched for ISCs at the bud tips and decrease in regions of differentiated cells at the base of the buds. To test this, we assessed Hmga1 protein levels throughout the organoids and found that Hmga1 is enriched at the bud tips where ISCs predominate, and undetectable in the differentiated cells, suggesting that differentiation is permitted in cells with lower levels of Hmga1 (FIG. 12). Together, these results indicate that Hmga1 is a key factor for ISC maintenance and self-renewal.

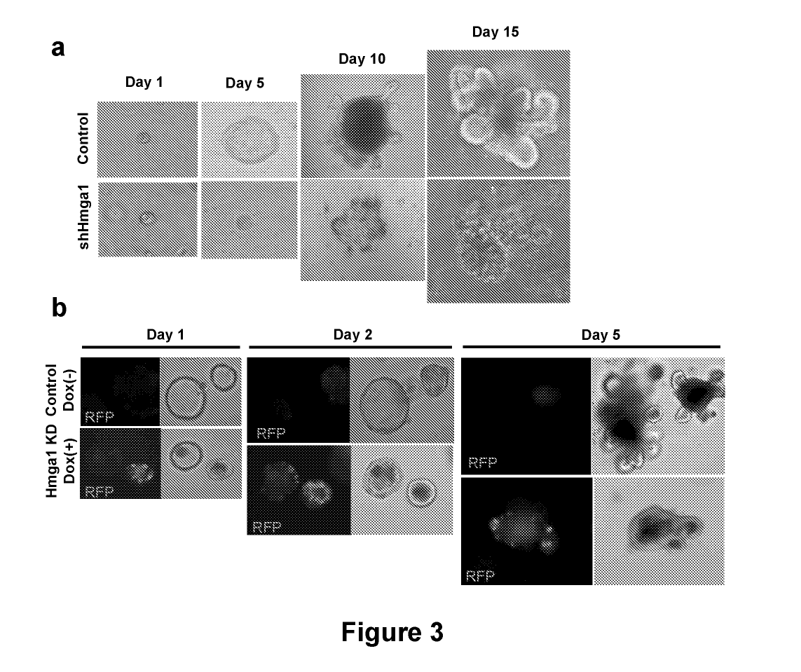

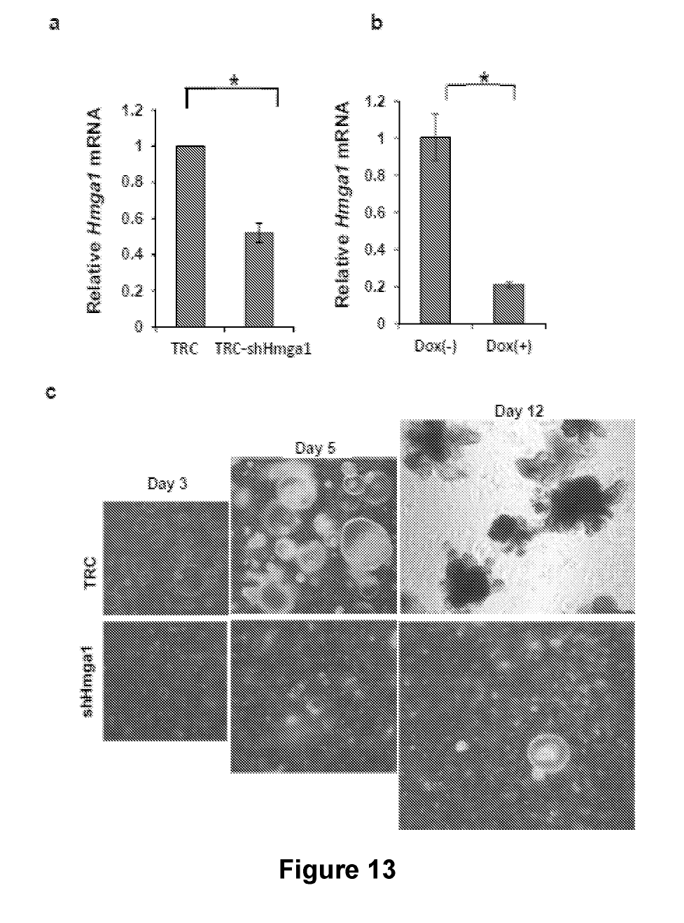

Because these studies demonstrate that Hmga1 enhances self-renewal when overexpressed in ISCs, we sought to determine whether it is required for self-renewal and ISC function. First, we silenced Hmga1 in WT crypt cells using lentiviral mediated delivery of shRNA targeting Hmga1 (shHmga1) and compared this to WT crypt cells transduced with a control lentiviral vector in organoid cultures. Crypt cells were incubated with Wnt3a (10 ng/ml) to enhance transduction efficiency as previously reported. Strikingly, the crypt cells transduced with the shHmga1 vector failed to reorganize into 3D structures and form buds, while those transduced with control vector organized into 3D structures and formed buds (FIG. 3a). To rule out any potential nonspecific toxicity from the shHmga1 vector, the inventors also tested an inducible, short hairpin RNA lentiviral vector targeting Hmga1 (inducible-shHmga1) that is labeled with red fluorescent protein (RFP) when shRNA is induced with doxycycline. Crypt cells transduced with the lentivirus, but not induced to express the shHmga1 vector, organized into 3D organoids with buds (FIG. 3b; top panel). In contrast, those organoids in which the shHMGA1 vector was induced did not organize into 3D organoid structures nor did they establish new buds following Hmga1 silencing (FIG. 3b; lower panel). Hmga1 gene silencing in organoids transduced with the constitutive or inducible shHMGA1 was confirmed by quantitative reverse transcription PCR (qRT-PCR; FIG. 13a-b). To further validate these findings, the inventors also transduced organoids derived from the Hmga1 mice with the shHmga1 vector and found that silencing Hmga1 disrupted their ability to form organoids and generate buds (FIG. 13c). These studies indicate that Hmga1 is required for self-renewal in ISCs and organization into 3D organoid structures.

### Hmga1 Induces ISC Self-Renewal Through Wnt/β-Catenin Signaling.

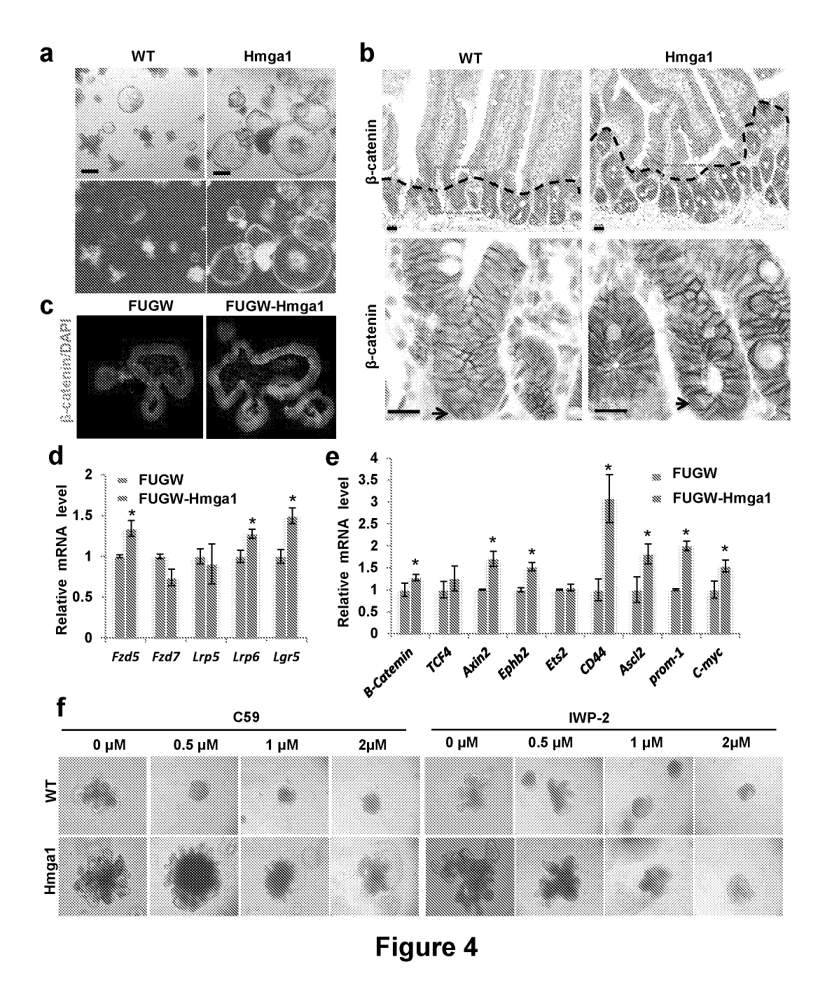

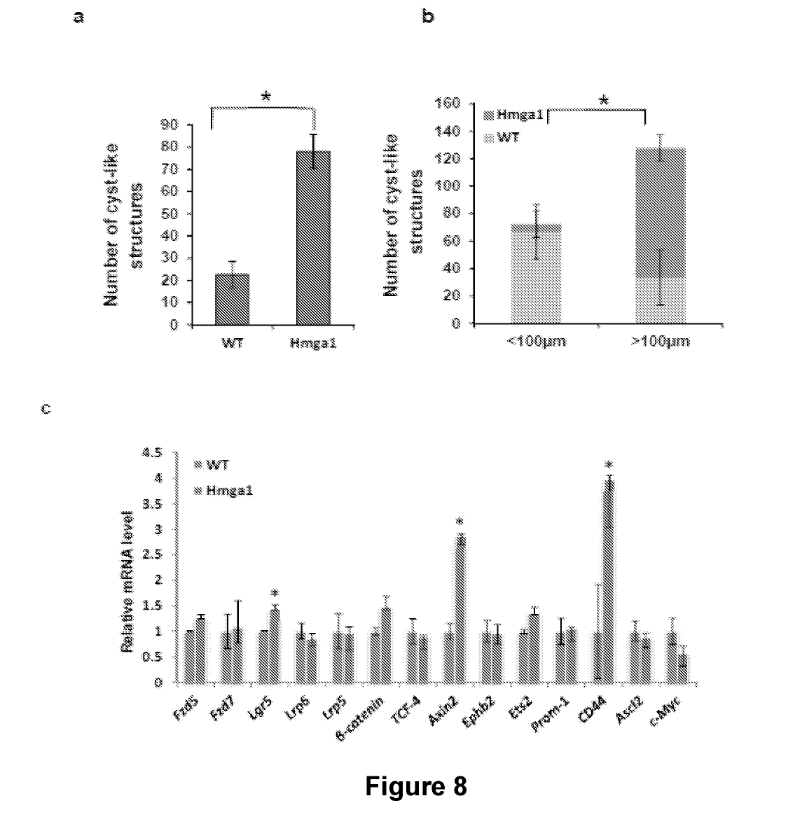

Surprisingly, when organoids from Hmga1 mice were incubated with Wnt3a to enhance lentiviral transduction, the inventors observed a dramatic phenotype whereby the organoids formed very large, cyst-like structures comprised predominantly of Lgr5+ cells, in contrast to WT organoids, which generated cysts that were significantly smaller and markedly decreased in number (FIG. 4a, FIG. 8a-b). These findings suggested that the Hmga1 organoids are more sensitive to Wnt signaling. Wnt/Tcf4/β-catenin signaling is an evolutionarily conserved pathway important for self-renewal in epithelial crypt ISCs and many other tissue-specific adult stem cells. Moreover, Wnt signaling activation leads to malignant transformation in intestinal epithelium. To begin to define the role of Hmga1 in Wnt/Tcf4/β-catenin signaling, we assessed immunostaining for β-catenin as a surrogate for canonical Wnt signaling. β-catenin was markedly increased in the Hmga1 transgenic intestinal epithelium and concentrated at the base of the crypts (FIG. 4b). A similar increase was also observed in the WT organoids transduced to overexpress Hmga1 (FIG. 4c). Since Hmga1 functions as an architectural transcription factor that alters gene expression, the inventors hypothesized that Hmga1 could up-regulate expression of factors that enhance Wnt signaling. To test this, the inventors first assessed expression of genes encoding Wnt agonist receptors that function in intestinal epithelium, including Lgr5, Frizzled (Fzd)5, Fzd7, low-density lipoprotein receptor-related protein 5 (Lrp5) and Lrp6. The inventors found a significant increase in expression of Lgr5, Fzd5, and Lrp6 in the organoids transduced to overexpress Hmga1 as compared to control organoids (FIG. 4d). Similarly, Lgr5 and Fzd5 were up-regulated in organoids from the Hmga1 transgenics as compared to WT organoids (FIG. 8c). Together, these results indicate that Hmga1 amplifies Wnt signaling by up-regulating genes encoding Wnt agonist receptors, including Lgr5. The basis for the variation in expression of genes encoding Wnt receptors induced by Hmga1 in the transgenic or transduced organoid model is not clear, although the crypt cells isolated from the transgenic mice developed in the setting of long-term, chronic overexpression of Hmga1, in contrast to organoids, which were transduced to overexpress higher levels of Hmga1 for shorter time periods. Nonetheless, both models demonstrate that Hmga1 enhances Wnt signaling by up-regulating Wnt agonist receptors.

Once β-catenin is released from an inhibitory complex following Wnt signaling, it binds to DNA together with its partner, Tcf4, to induce Wnt pathway genes. In human embryonic stem cells, HMGA1 induces expression of c-MYC, a WNT/TCF4/β-catenin gene target, suggesting that Hmga1 could cooperate with Wnt/Tcf4/β-catenin to regulate the Wnt stem cell program. We therefore assessed expression of Wnt/Tcf4/β-catenin target genes, including Axin2, Ascl2, β-catenin, CD44 c-Myc Ephb2, Ets, Prom-1, and Tcf4. Expression of all of these genes, excluding Ets, was significantly increased in WT organoids transduced to overexpress Hmga 1 as compared to control organoids (FIG. 4e). β-catenin, Axin2, Ets2, and CD44 were also induced in organoids from the Hmga 1 transgenic mice as compared to WT mice (FIG. 8c). Together, these results indicate that Hmga1 not only activates Wnt target genes together with Tcf4/β-catenin, but also amplifies Wnt signals by inducing expression of Wnt agonist receptor genes.

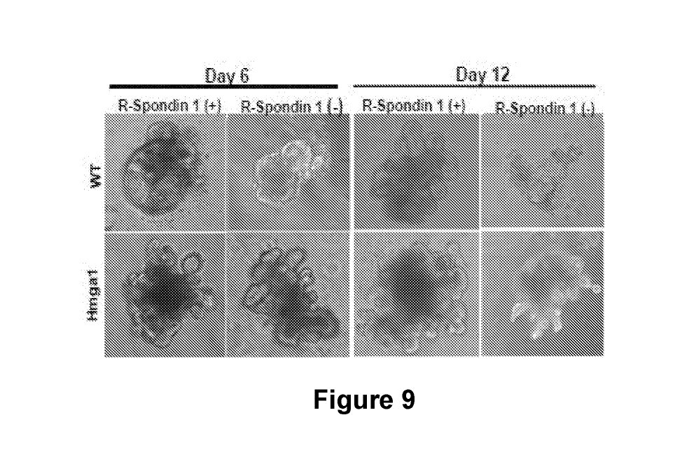

To better define the role of Hmga1 in regulating Wnt signaling, we cultured Hmga1 organoids in the absence of the Wnt receptor agonist, R-spondin 1 (R-spo1), which is essential for organoid formation in this culture system. R-spo1 is secreted by intestinal stromal cells and binds to the Lgr5 receptor to activate Wnt signaling. The inventors discovered a dramatic difference in the response of Hmga1 organoids to the absence of R-spo1 as compared to the WT controls: Hmga1 organoids continued to survive and proliferate after 2 weeks, while the WT organoids lost their 3D structural organization and ultimately died by day 5 (FIG. 9). By 3 weeks, the Hmga1 organoids also stopped proliferating and lost their 3D organization, suggesting that Hmga1 partially rescues loss of Wnt signaling via R-spo1. To further test the link between Hmga1 and Wnt, we treated organoids with the Wnt inhibitors, C59 and IWP-2, both of which block Wnt-mediated transcription and cell proliferation by inhibiting porcupine (PORCN), a protein required for Wnt palmitoylation, secretion, and biological activity. The Hmga1 organoids were able to survive in up to 2μM of C59, while the WT controls failed to proliferate with exposure to as little as 0.5 μM (FIG. 4f). Similarly, the Hmga1 organoids survive and form buds in 1 μM of IWP-2, whereas controls form few buds at 0.5 μM and no buds at 1 μM IWP-2 (FIG. 4f). The blunted response of Hmga1 organoids to Wnt inhibition further highlights the ability of Hmga1 to amplify Wnt signaling to maintain and support ISCs.

### Hmga1 Expands the Paneth Cell Niche Through Sox9.

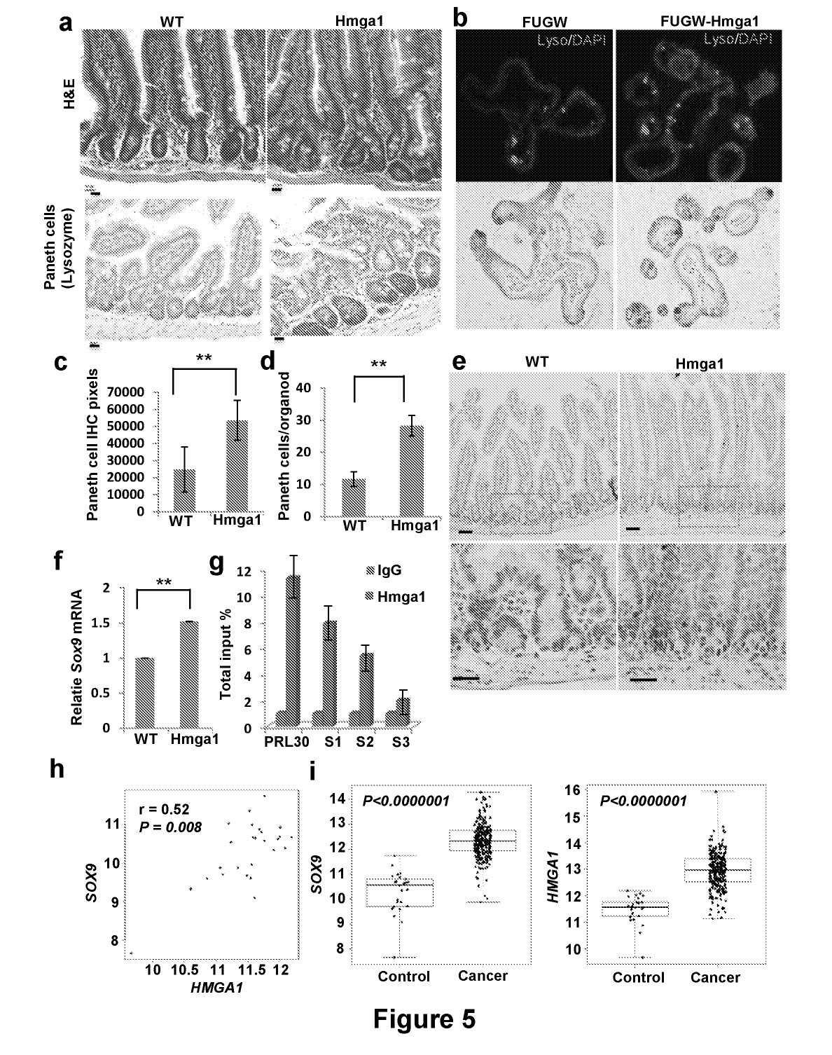

Paneth cells are terminally differentiated epithelial cells derived from ISCs and located at the base of intestinal crypts. They support ISC survival by secreting Wnt3a and other factors, thus providing an epithelial niche for ISCs. To determine whether Hmga1 alters the Paneth cell niche in our transgenic models, we stained for lysozyme using alkaline phosphatase, which marks Paneth cells (FIG. 5a-d). Total Paneth cell number per unit area was assessed quantitatively in both mouse tissues and organoids (IHC pixels via imaginepro-plus 6.0 software). Surprisingly, there was a marked expansion in the Paneth cell niche in the Hmga1 transgenic small intestinal epithelium as compared to WT control intestine (FIG. 5a, c). Paneth cells were also increased in WT organoids transduced to overexpress Hmga1 (FIG. 5b; FUGW-Hmga1). Together, these results indicate that Hmga1 induces Paneth cell niche formation and expansion, which could promote ISC maintenance and expansion.

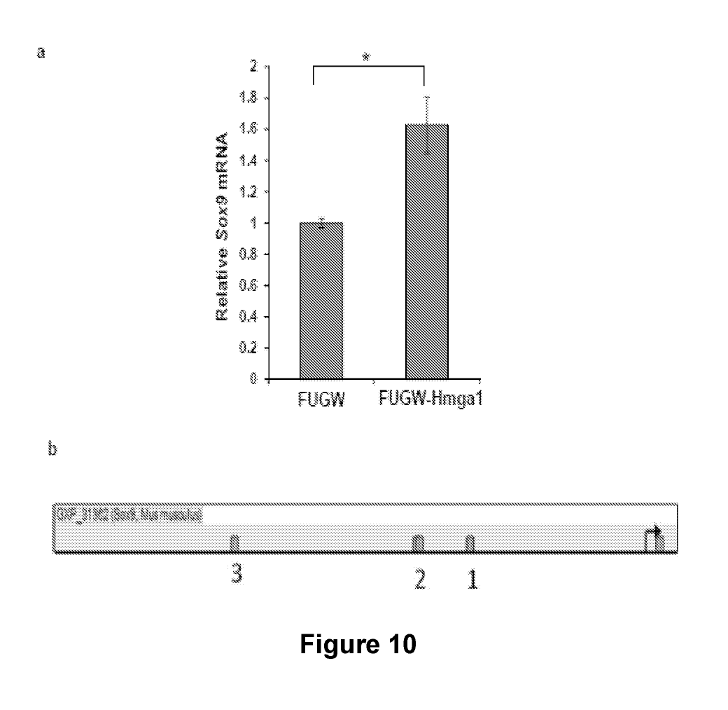

Because terminal differentiation to a Paneth cell requires Sox9, the inventors hypothesized that Hmga1 induces Paneth cell expansion by up-regulating Sox9 expression. HMGA1 induces the SOX family member, SOX2, in human embryonic stem cells; further, both human and mouse SOX9/Sox9 have similar AT-rich regions and predicted Hmga1 DNA binding sites in the 5′ untranslated region. Sox9 is also a β-catenin/Tcf4 target gene. The inventors found that Hmga1 up-regulates Sox9 protein levels in transgenic small intestinal tissue as compared to WT tissues (FIG. 5e). Sox9 mRNA was also increased in organoids derived from the Hmga1 transgenic mouse model as compared to WT mice (FIG. 5f) and in WT organoids engineered to overexpress Hmga1 (FIG. 10). To determine whether Hmga1 directly induces Sox9, the inventors performed chromatin immunoprecipitation (ChIP) in intestinal crypt cells from WT mice. The inventors discovered that Hmga1 binds directly to the Sox9 promoter at 3 conserved sites (FIG. 5g; FIG. 10b). Enrichment of Hmga1 binding is greatest at the two more distal sites, and significant, albeit lower, at the proximal site. Together, the results reveal a novel role for Hmga1 in establishing an epithelial stem cell niche through Paneth cell differentiation in addition to driving self-renewal and expansion of ISCs.

### HMGA1 and SOX9 are Highly Correlated in Human Intestine and Up-Regulated in Colorectal Cancer.

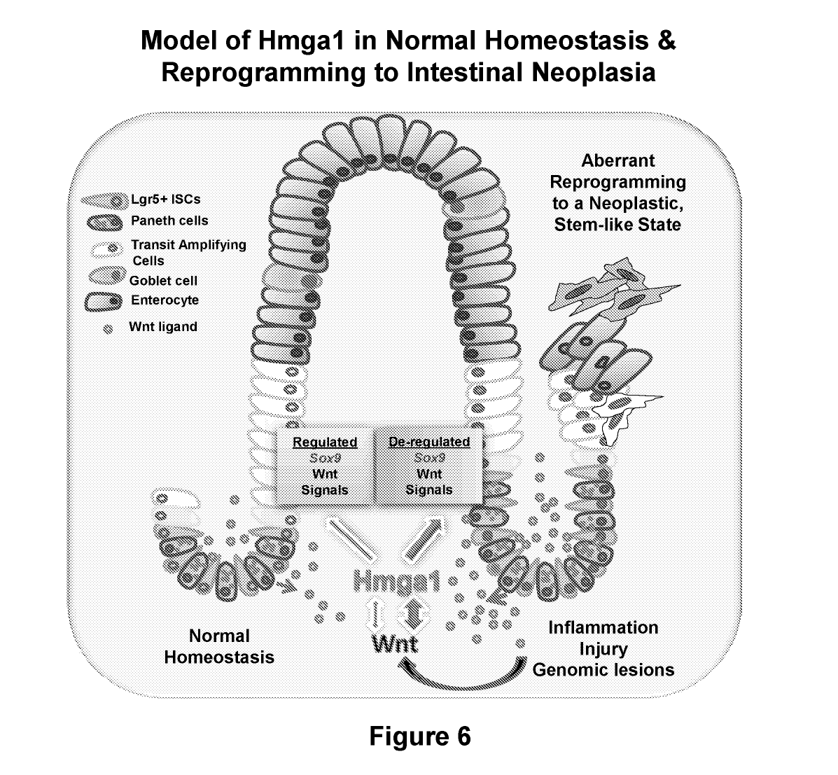

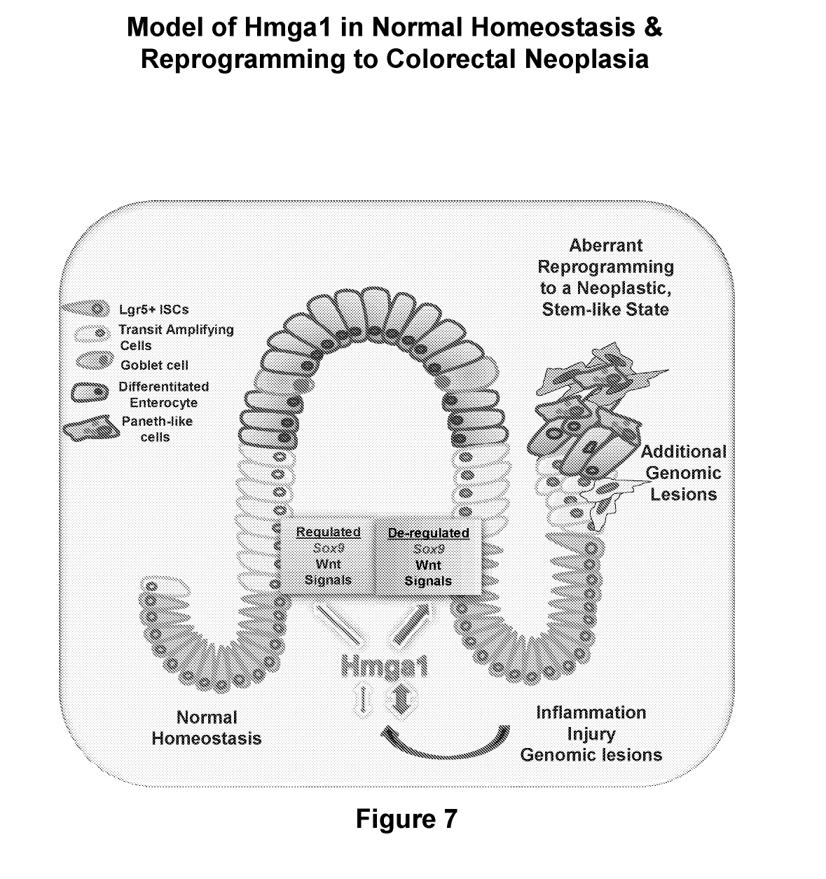

To determine if the above findings in mouse intestine are relevant to humans, the inventors assessed expression of HMGA1 and SOX9 in human intestinal epithelium. Using the Cancer Genome Atlas (TCGA), the inventors found that HMGA1 and SOX9 are positively correlated in normal colonic epithelium (P=0.008, r=0.52; FIG. 5e). The inventors also stained human small intestine, and found that HMGA1 localizes to the columnar basal cells within the crypts. Because HMGA1 is overexpressed in diverse epithelial cancers and correlates with cancer stem cell properties in experimental models, the inventors also sought to determine whether HMGA1 and SOX9 are co-regulated in colorectal cancer. Strikingly, both HMGA1 and SOX9 are significantly up-regulated in human colorectal cancer (P<0.0000001), although their expression was not correlated in this setting (FIG. 5h-i). Interestingly, the relative expression and amplitude of HMGA1 mRNA levels are greater than that of SOX9 (FIG. 5h-i). These data support a model whereby HMGA1 and SOX9 are crucial for normal ISC function, and both become up-regulated in carcinogenesis (FIG. 6). These findings also suggest that up-regulation of both HMGA1 and SOX9 beyond a threshold may be necessary for early reprogramming of an epithelial cell to a neoplastic cell, while further increases in HMGA1 could drive tumor progression. Of note, a prior study in colorectal cancer found HMGA1 to be among the genes most enriched in cancer relative to adjacent, nonmalignant tissue.

HMGA1 expression has been identified among genes most enriched in embryonic and adult stem cells, although its function in these settings had been poorly understood. The present invention revealed a novel role for Hmga1 in both stem cell self-renewal and establishment of a stem cell niche within small intestinal crypts. The HMGA gene family includes HMGA1 (on chromosome 6p21) and HMGA2 (on chromosome 12q15), both of which are highly expressed during embryonic development, but with low or undetectable levels in differentiated tissues. Aberrant expression of HMGA1 occurs in most poorly differentiated human cancers, including gastrointestinal cancers such as colon, gastric, pancreatic, and esophageal cancers, and high levels correlate with poor outcomes in diverse tumors. HMGA1 is also required for properties attributed to cancer stem cells, including tumor initiator cells, growth as 3D spheres, and metastatic progression. In contrast, HMGA2 overexpression occurs primarily in benign tumors of mesenchymal origin. The dual role for Hmga1 in normal development and poorly differentiated cancers suggests that it regulates cell fate decisions, although a detailed understanding of molecular mechanisms involved in these processes was previously unknown.

Here, the inventors show for the first time that Hmga1 amplifies Wnt signaling to drive self-renewal and ISC expansion. Hmga1 not only up-regulates Wnt agonist receptor genes, but also enhances expression of genes downstream of WntTcf4/β-catenin. Our results, together with the prior finding that Tcf4 binds to the HMGA1 promoter in colorectal cancer cells, suggest that Hmga1 is involved in a “feed-forward” loop whereby Tcf4/β-catenin induces Hmga1, leading to enhanced Wnt signaling. The significant expansion of ISCs in our transgenic mouse model was recapitulated in vitro in organoid cultures, which depend on Hmga1 for organization into 3D structures and bud formation. The inventor's transgenic mouse and organoid models provide valuable tools to further dissect downstream pathways regulated by Hmga1 in ISCs. In a murine model of gastric cancer, Wnt signaling also up-regulates Hmga1. The inventors are the first to show that Hmga1 enhances Wnt/β-catenin signaling at multiple levels in the pathway, consistent with a feed-forward amplification loop whereby Wnt induces Hmga1, which in turn, up-regulates Wnt/β-catenin signaling (FIG. 6).

Our work also uncovered an unexpected and unique role for Hmga1 in Paneth cell differentiation through Sox9. Paneth cells constitute an epithelial niche, providing Wnt3a and other signals to maintain ISCs and permit self-renewal within the crypts. Cues from the epithelial and stromal niche are likely to help govern whether Hmga1 induces Sox9 to drive Paneth cell differentiation or Wnt signaling to drive self-renewal. Because adult stem cells can divide asymmetrically, Hmga1 could also promote ISC division to generate both an identical daughter cell and a differentiated Paneth cell, or even a more fully differentiated transit-amplifying cell. Recent studies indicate that R-spondin 1, another Wnt receptor agonist secreted by intestinal stromal cells, is required for ISC maintenance in mice. It remains to be seen whether Hmga1 also regulates R-spondin 1 in stromal niche cells. Of note, mesenchymal stem cells within the bone marrow niche express high levels of Hmga1 and preliminary studies suggest that Hmga1 is required by mesenchymal stem cells to secrete factors that support the survival of hematopoietic stem cells.

In addition to establishing a role for Hmga1 in maintaining ISC and niche compartments, we also found a highly significant correlation between HMGA1 and SOX9 in normal human large intestinal epithelium. Moreover, both genes are up-regulated in colorectal cancer, indicating that this pathway may contribute to human intestinal carcinogenesis. A recent study in the Adenomatous Polyposis Coli (Apc)^{+/-} murine model of intestinal carcinogenesis showed that Hmga1 is downstream of the miR-26 tumor suppressor, suggesting that APC mutations in colorectal cancer lead to HMGA1 induction. HMGA1 is also up-regulated in the setting of inflammation, and intestinal carcinogenesis is frequently preceded by chronic inflammation and injury. Thus, both inflammatory lesions and genetic alterations (APC inactivating mutations) could cooperate to induce HMGA1 during carcinogenesis. Paneth cell expansion also occurs in mice with deletion in the Apc tumor suppressor, which may depend on Hmga1. Although there are no discrete populations of Paneth cells outside of the proximal colon in humans, lysozyme-expressing Paneth-like cells are found in human adenomas. Based on our findings, it is plausible that HMGA1 induces formation of Paneth-like niche cells during carcinogenesis in the intestinal epithelium. Studies in murine pancreatic cancer show that SOX9 reprograms acinar cells to ductal cells during carcinogenesis. Similarly, HMGA1 could induce SOX9 and enforce Wnt signaling to drive stem cell properties and reprogram intestinal epithelial cells during carcinogenesis. Thus, our work not only reveals a novel function for Hmga1 in intestinal homeostasis through self-renewal of ISCs and Paneth cell differentiation, but also sheds light on the mechanisms involved in Hmga1-mediated neoplastic transformation and intestinal carcinogenesis.

### EXAMPLES/METHODS

Mouse models. The Hmga1 transgenic construct and mice have been previously described. Female Lgr5-eGFP-IRES-CreERT2 mice⁶ (Jackson Labs) were crossed with male Hmga1a transgenics. All animal experiments were conducted in accordance with our institutional Animal Care and Use Committee (protocol #MO14M187). All mice were housed in a sterile environment where they had free access to food and water as outlined in our institutional guidelines.

Crypt isolation. Crypts were isolated as previously described. After isolation, crypt cells were pelleted, passed through a 70 μm cell strainer, evaluated for purity microscopically, and counted.

Organoid culture and replating assay. Mouse organoids were established and maintained from isolated crypts of the proximal small intestine as described previously¹⁻⁷. The basic culture medium (advanced Dulbecco's modified Eagle's medium/F12 supplemented with penicillin/streptomycin, 10 mmol/l HEPES, 13 Glutamax, 13 B27 [all from Life Technologies], and 1 mmol/l N-acetylcysteine [Sigma]) was supplemented with 50 ng/ml murine recombinant EGF (Peprotech), R-spondin 1 (1 μg/ml), and Noggin (10 ng/ml). Wnt inhibitors C59 and IWP-2 are commercially available (Abcam). Conditioned media was produced using HEK293T cells stably transfected with HA-mouse Rspo1-Fc (a gift from Calvin Kuo, Stanford University). Advanced DMEM/F12 supplemented with penicillin/streptomycin, 10 mmol/1 HEPES, and 13 Glutamax was conditioned for 1 week. For the replating assay, an equal number of organoids from each model were mechanically dispersed with a 10 mL pipet after 3-5 days in culture and replated using the conditions described above.

Lentivirus and transduction. The FUGW and FUGW-Hmga1 lentiviral vectors^{8, 9} and the short hairpin RNA (shRNA) interference plasmids and control vector have been described. For inducible silencing, pTRIPz-Hmga1-shRNA linked to red florescence protein (RFP) reporter was generated with inducible expression with doxcycline (0.5 μg/ml). We modified an established protocol using magnetic nanoparticles (ViroMag R/L, OZ Bioscience, Inc) and a magnetic plate (ViroMag R/L, OZ Bioscience, Inc, catalog number: MF10000) to transduce crypt cells and organoids.

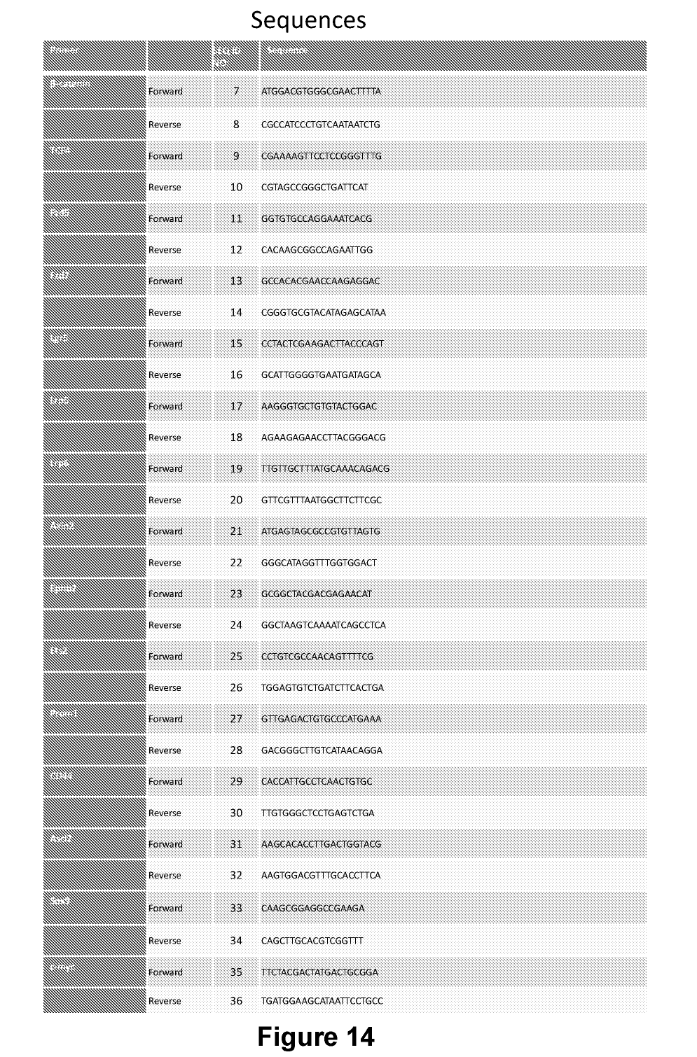

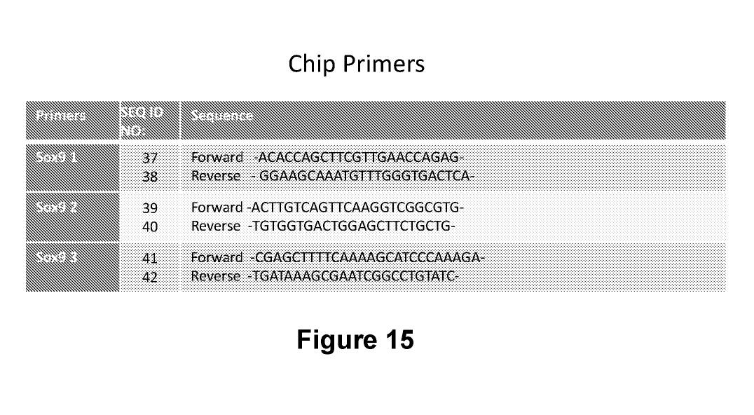

Gene expression analysis and chromatin immunoprecipiation (ChIP). Wnt signaling molecule gene expression was detected by qRT-PCR as we described. (See primers in FIG. 14). For ChIP experiments, primers were designed using the sequence data from MatInspector in silico transcription factor binding site prediction algorithm with amplicon sizes of around 200 base pairs as we described. (See FIG. 15 for ChIP primer sequences.)

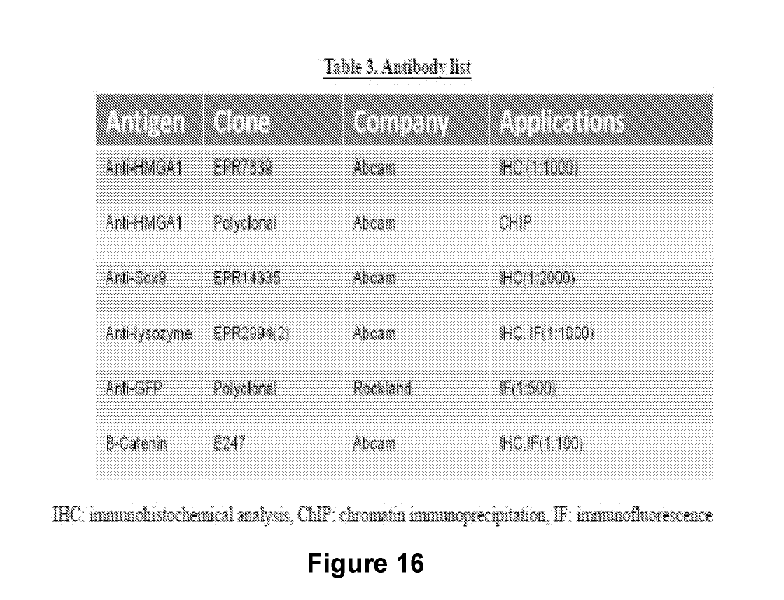

Immunohistochemistry. Hematoxylin & Eosin (H&E) and immunohistochemistry (IHC) staining of organoid and intestinal sections were performed as previously described (See for details; primary antibodies are listed in FIG. 16).

Crypt Isolation. Crypts were isolated as previously described. Briefly, intestines were flushed with phosphate buffered saline (PBS) and incised longitudinally after which villi were removed mechanically by scraping. Sections (1 cm) were incubated in EDTA (5 mM)/PBS for 15 minutes at 4° C. per fraction of epithelium. After incubation, the epithelium was separated by vigorous shaking, and the remaining intestinal tissue was placed in a new tube for collection of subsequent fractions. After isolation, crypt cells were pelleted, passed through a 70 μm cell strainer, and evaluated for purity microscopically.

Lentivirus and Transduction. The short hairpin RNA (shRNA) interference plasmid for Hmga1 (#TRCN0000182169; the RNAi Consortium/TRC) have been described. The empty shRNA vector was used as a control. pTRIPz-Hmga1-shRNA was engineered to be inducible by tetracycline or analogues (tet-On) and produces tightly regulated induction of shRNA expression in the presence of doxycycline. The annealed Hmga1-shRNA oligonucleotides were cloned into linearized pTRIPZ empty vector (Open Biosystems catalog #RHS4750). The FUGW-GFP plasmid was a gift from David Baltimore (Addgene plasmid # 14883). Hmga1-FUGW was previously described. For lentiviral transduction, the inventors used magnetic medium that was previously reported. Organoid fragments were seed with 150 ul transduction medium into 48-well plates. Virus was added with viroMag R/L solution 15 min at RT (2500-3000virus particles/cells) to the cells to be transduced. The cell culture plate was placed on the magnetic plate for 30-60 minutes in a 37° C. tissue culture incubator. Cells were then incubated overnight at 37° C. The organoid fragments and transduction media were then transferred to a 1.5 ml tube for centrifugation at 900 x g for 5 minutes. The supernatant was discarded and the tube containing the pellet was placed on ice for 5 minutes. Next, 120 μl of matrigel was added and the pellet was resuspended by pipetting slowly up and down. Drops (30p1) of basement matrix-cell mixtures were seeded into a new 48-well plate and incubated at 37° C. for 5-15 min until the basement matrix solidified. ENRWntNic medium was then added to the wells and placed into a tissue culture incubator. Common ENR media was used and changed every 2-3 days 4-6 days after the transduction. Selection media was added after 2-3 days by adding puromycin (2 μg/ml).

Gene Expression Analysis and Chromatin Immunoprecipitation (ChIP). Wnt signaling molecule gene expression was detected by qRT-PCR as described previously. Primers are listed in Table 1 (FIG. 14) Reactions were performed using the SYBR Green PCR Master Mix (Applied Biosystems) with the ABI 7500 qRT-PCR machine. Gapdh mRNA was assessed as the loading control. For validation of ChIP experiments, the enriched peaks were confirmed using qRT-PCR. Primers were designed using the sequences data from MatInspector in silico transcription factor binding site prediction algorithm with amplicon sizes of around 200 base pairs. Precipitated DAN was purified and diluted d 1:20 before testing with qRT-PCR. The C (t) values were quantified using a standard made by serial dilutions of ChIP input material. Values were normalized against the corresponding input material and, finally, against a negative control region. Primer sequences for ChIP experiments are listed in Table 2 (FIG. 15).

Immunohistochemistry. Organoid cultures were fixed overnight in formalin at 4° C. before paraffin or frozen embedding. H&E and immunohistochemistry staining on organoid and intestinal sections were performed as previously described, the primary antibodies are listed in Table 3 (FIG. 16).
1. Sato, T., Clevers, H. Growing self-organizing mini-guts from a single intestinal stem cell: mechanisms and application. Science 340, 1190-1194 (2013).
2. Koo, B-K, Clevers, H. Stem cells marked by the R-Spondin receptor Lgr5. Gastroenterology 147, 289-302 (2014).

3. Barker, N. van Es, J. H., Kuipers, J., Kujala P, van den Born M, et al. Identification of stem cells in small intestine and colon by marker gene Lgr5. Nature 449, 1003-1007 (2007).
4. Schuijers, J., Junker, J., Mokry, M., Hatzis, P., Koo, B., et al. Ascl2 acts as an R-spondin/Wnt-responsive switch to control sternness in intestinal crypts. Cell Stem Cell. 16, 158-170 (2015).
5. Cheung, E. C., Athineos, D., Lee, P., Ridgway, R. A., Lambie, W., et al. TIGAR is required for efficient intestinal regeneration and tumorigenesis. Dev Cell 25, 463-477 (2013)
6. Sato, T., Clevers, H. Primary mouse small intestinal epithelial cell cultures. Methods Mol Biol 945, 319-328 (2013).
7. Andersson-Rolf, A., Fink, J., Mustata, R. C., Koo, B-K. A video protocol of retroviral infection in primary intestinal organoid culture. J Vis Exp 11, e51765 (2014).
8. Tesfaye, A., Di Cello, F., Hillion, J., Ronnett, B. M., Elbahloul, O., et al. The high-mobility group A1 gene up-regulates cyclooxygenase 2 expression in uterine tumorigenesis. Cancer Res 67, 3998-4004 (2007).
9. Lois C, Hong E J, Pease S, Brown E J, Baltimore D. Germline transmission and tissue-specific expression of transgenes delivered by lentiviral vectors. Science 295, 868-872 (2002).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing a genetically modified organoid comprising the following steps:
a. isolating a crypt of the intestine;
b. culturing at least a portion of the crypt in a culture medium;
c. forming an organoid; and
d. transducing the organoid with a vector expressing a Hmga1 protein or functional part thereof.

2. The method of claim 1, wherein the vector is a lentiviral vector.

3-4. (canceled)

5. The method of claim 1, wherein at least portion of the crypt includes intestinal stem cells.

6. The method of claim 1, wherein the genetically modified organoid overexpresses Hmga1 protein, or a functional part thereof, when compared to a second organoid substantially free of a vector expressing the Hmga1 protein or functional part thereof.

7. The method of claim 1, wherein the Hmga 1 protein is a human Hmga 1 protein.

8. The method of claim 1, wherein the vector is selected from the group consisting of retroviruses, lentiviruses, adenoviruses, adeno-associated viruses, or a combination thereof.

9. A genetically modified organoid comprising:
a. a cell of the intestine; and
b. a vector expressing a Hmga1 protein or functional part thereof in the cell.

10. The genetically modified organoid of claim 9 wherein the vector is integrated in the genome of the cell.

11. The genetically modified organoid of claim 9 having enhanced Wnt signaling when comparing the Wnt signaling of a second organoid substantially free of a vector expressing a Hmga 1 protein or a functional part thereof.

12. The genetically modified organoid of claim 9 wherein the enhanced Wnt signaling enhances expression of genes downstream of WntTcf4/β-catenin.

13. The genetically modified organoid of claim 9 comprising increased numbers of Paneth when compared to a reference organoid substantially free of a vector expressing a Hmga 1 protein or functional part thereof.

14. A genetically modified organoid of claim 9 wherein the genetically modified organoid comprises Sox 9 and the Hmga1 protein expressed from the vector up-regulates Sox 9 expression.

15. A method of treating or preventing an injury in a subject comprising the following steps:
a. providing a subject with an intestinal injury; and
b. placing an organoid comprising a vector expressing a Hmga1 protein, or functional part thereof, adjacent to the site of the intestinal injury.

16. The method of claim 15 wherein the organoid comprises an intestine cell, the vector is a lentiviral vector, and the lentiviral vector is inserted in a genome of the intestine cell.

17. The method of claim 15 wherein there the organoid has an increased number of Paneth when compared to a reference organoid substantially free of a vector expressing a Hmga 1 protein, or functional part thereof.

18. The method of claim 15 wherein the subject has enhanced intestinal stem cell maintenance compared to a reference subject that has not undergone the method of claim 11.

19. The method of claim 15 wherein the subject has been administered chemotherapy.

20. The method of claim 15 wherein the subject has been administered radiation.

21. The method of claim 15 wherein there is an increase number of Paneth adjacent to the site of the intestinal injury.

22. A method of screening a pharmaceutical agent comprising:
a. providing a first organoid comprising a vector expressing a Hmga1 protein or functional part thereof;
b. applying an agent to the first organoid; and
c. identifying those agents that inhibit the expression or activity of the Hmga1 protein, or functional part thereof, in the first organoid.

23. The method of claim 22 wherein the one or more agents is selected from a group consisting of a chemical, protein, peptide, nucleic acid sequence, or combination thereof.

24. The method of claim 22 further providing a reference organoid that is substantially free of a vector expressing a Hmga 1 protein, or functional part thereof, and the agent is applied to the reference organoid.

25. A method of treating or preventing intestinal cancer in a subject comprising administering to the subject an effective amount of a Hmga1 inhibitor or a pharmaceutically acceptable salt, solvate, or stereoisomer thereof.

26. The method of claim 25 wherein the Hmga1 inhibitor is selected from the group consisting of a chemical, peptide, an antibody, shRNA, nucleic acid, or combination thereof.

27. The method of claim 25 wherein the intestinal cancer is colorectal neoplasia.


SEQUENCE LISTING (42 sequences)

SEQ ID NO: 1 - DNA, 324 bp, Homo sapiens
atgagtgagtcgagctcgaagtccagccagcccttggcctccaagcaggaaaaggacggcactgagaagcggggccggggcaggccgcgcaagcagcctccggtgagtcccgggacagcgctggtagggagtcagaaggagcccagcgaagtgccaacacctaagagacctcggggccgaccaaagggaagcaaaaacaagggtgctgccaagacccggaaaaccaccacaactccaggaaggaaaccaaggggcagacccaaaaaactggagaaggaggaagaggagggcatctcgcaggagtcctcggaggaggagcagtga

SEQ ID NO: 2 - protein, 107 aa, Homo sapiens
MSESSSKSSQPLASKQEKDGTEKRGRGRPRKQPPVSPGTALVGSQKEPSEVPTPKRPRGRPKGSKNKGAAKTRKTTTTPGRKPRGRPKKLEKEEEEGISQESSEEEQ

SEQ ID NO: 3 - DNA, 321 bp, Mus musculus
atgagcgagtcgggctcaaagtccagccagcccctggcctccaaacaggaaaaggatgggactgagaagcgaggccggggcaggccacgcaagcagcctccggtgagtcctgggacggcgctggtcgggagtcagaaagagcccagtgaagtgccaactccgaagagacctcggggccgaccaaagggaagcaagaataagggcgccgccaagacccggaaagtcaccacagctccagggaggaaaccaaggggcagacccaagaaactggagaaggaggaagaggagggcatctcccaggagtcctctgaggaggagcag

SEQ ID NO: 4 - protein, 107 aa, Mus musculus
MSESGSKSSQPLASKQEKDGTEKRGRGRPRKQPPVSPGTALVGSQKEPSEVPTPKRPRGRPKGSKNKGAAKTRKVTTAPGRKPRGRPKKLEKEEEEGISQESSEEEQ

SEQ ID NO: 5 - DNA, 59 bp, Artificial Sequence
  Description of Artificial Sequence Synthetic oligonucleotide
ccggcccactttaatgttctcacctctcgagaggtgagaacattaaagtgggttttttg

SEQ ID NO: 6 - DNA, 97 bp, Artificial Sequence
  Description of Artificial Sequence Synthetic oligonucleotide
tgctgttgacagtgagcgagctccctctctggtttcctattagtgaagccacagatgtaataggaaaccagagagggagcctgcctactgcctcgga

SEQ ID NO: 7 - DNA, 21 bp, Artificial Sequence
  Description of Artificial Sequence Synthetic primer
atggacgtgggcgaactttta

SEQ ID NO: 8 - DNA, 22 bp, Artificial Sequence
  Description of Artificial Sequence Synthetic primer
cgccatccctgtcaataatctg

SEQ ID NO: 9 - DNA, 21 bp, Artificial Sequence
  Description of Artificial Sequence Synthetic primer
cgaaaagttcctccgggtttg

SEQ ID NO: 10 - DNA, 19 bp, Artificial Sequence
  Description of Artificial Sequence Synthetic primer
cgtagccgggctgattcat

SEQ ID NO: 11 - DNA, 19 bp, Artificial Sequence
  Description of Artificial Sequence Synthetic primer
ggtgtgccaggaaatcacg

SEQ ID NO: 12 - DNA, 19 bp, Artificial Sequence
  Description of Artificial Sequence Synthetic primer
cacaagcggccagaattgg

SEQ ID NO: 13 - DNA, 20 bp, Artificial Sequence
  Description of Artificial Sequence Synthetic primer
gccacacgaaccaagaggac

SEQ ID NO: 14 - DNA, 22 bp, Artificial Sequence
  Description of Artificial Sequence Synthetic primer
cgggtgcgtacatagagcataa

SEQ ID NO: 15 - DNA, 22 bp, Artificial Sequence
  Description of Artificial Sequence Synthetic primer
cctactcgaagacttacccagt

SEQ ID NO: 16 - DNA, 21 bp, Artificial Sequence
  Description of Artificial Sequence Synthetic primer
gcattggggtgaatgatagca

SEQ ID NO: 17 - DNA, 20 bp, Artificial Sequence
  Description of Artificial Sequence Synthetic primer
aagggtgctgtgtactggac

SEQ ID NO: 18 - DNA, 21 bp, Artificial Sequence
  Description of Artificial Sequence Synthetic primer
agaagagaaccttacgggacg

SEQ ID NO: 19 - DNA, 23 bp, Artificial Sequence
  Description of Artificial Sequence Synthetic primer
ttgttgctttatgcaaacagacg

SEQ ID NO: 20 - DNA, 22 bp, Artificial Sequence
  Description of Artificial Sequence Synthetic primer
gttcgtttaatggcttcttcgc

SEQ ID NO: 21 - DNA, 21 bp, Artificial Sequence
  Description of Artificial Sequence Synthetic primer
atgagtagcgccgtgttagtg

SEQ ID NO: 22 - DNA, 20 bp, Artificial Sequence
  Description of Artificial Sequence Synthetic primer
gggcataggtttggtggact

SEQ ID NO: 23 - DNA, 19 bp, Artificial Sequence
  Description of Artificial Sequence Synthetic primer
gcggctacgacgagaacat

SEQ ID NO: 24 - DNA, 22 bp, Artificial Sequence
  Description of Artificial Sequence Synthetic primer
ggctaagtcaaaatcagcctca

SEQ ID NO: 25 - DNA, 20 bp, Artificial Sequence
  Description of Artificial Sequence Synthetic primer
cctgtcgccaacagttttcg

SEQ ID NO: 26 - DNA, 22 bp, Artificial Sequence
  Description of Artificial Sequence Synthetic primer
tggagtgtctgatcttcactga

SEQ ID NO: 27 - DNA, 21 bp, Artificial Sequence
  Description of Artificial Sequence Synthetic primer
gttgagactgtgcccatgaaa

SEQ ID NO: 28 - DNA, 21 bp, Artificial Sequence
  Description of Artificial Sequence Synthetic primer
gacgggcttgtcataacagga

SEQ ID NO: 29 - DNA, 20 bp, Artificial Sequence
  Description of Artificial Sequence Synthetic primer
caccattgcctcaactgtgc

SEQ ID NO: 30 - DNA, 20 bp, Artificial Sequence
  Description of Artificial Sequence Synthetic primer
ttgtgggctcctgagtctga

SEQ ID NO: 31 - DNA, 21 bp, Artificial Sequence
  Description of Artificial Sequence Synthetic primer
aagcacaccttgactggtacg

SEQ ID NO: 32 - DNA, 21 bp, Artificial Sequence
  Description of Artificial Sequence Synthetic primer
aagtggacgtttgcaccttca

SEQ ID NO: 33 - DNA, 17 bp, Artificial Sequence
  Description of Artificial Sequence Synthetic primer
caagcggaggccgaaga

SEQ ID NO: 34 - DNA, 18 bp, Artificial Sequence
  Description of Artificial Sequence Synthetic primer
cagcttgcacgtcggttt

SEQ ID NO: 35 - DNA, 21 bp, Artificial Sequence
  Description of Artificial Sequence Synthetic primer
ttctacgactatgactgcgga

SEQ ID NO: 36 - DNA, 22 bp, Artificial Sequence
  Description of Artificial Sequence Synthetic primer
tgatggaagcataattcctgcc

SEQ ID NO: 37 - DNA, 23 bp, Artificial Sequence
  Description of Artificial Sequence Synthetic primer
acaccagcttcgttgaaccagag

SEQ ID NO: 38 - DNA, 24 bp, Artificial Sequence
  Description of Artificial Sequence Synthetic primer
ggaagcaaatgtttgggtgactca

SEQ ID NO: 39 - DNA, 24 bp, Artificial Sequence
  Description of Artificial Sequence Synthetic primer
acttgtcagttcaaggtcggcgtg

SEQ ID NO: 40 - DNA, 23 bp, Artificial Sequence
  Description of Artificial Sequence Synthetic primer
tgtggtgactggagcttctgctg

SEQ ID NO: 41 - DNA, 26 bp, Artificial Sequence
  Description of Artificial Sequence Synthetic primer
cgagcttttcaaaagcatcccaaaga

SEQ ID NO: 42 - DNA, 24 bp, Artificial Sequence
  Description of Artificial Sequence Synthetic primer
tgataaagcgaatcggcctgtatc